(19) United States
(12) Patent Application Publication
(10) Pub. No.: US 2002/0052634 A1
(43) Pub. Date: May 2, 2002

(54) Method and system for modulation of oscillating signals to enhance biologic effects

(76) Inventor: Keith L. March, Carmel, IN (US)

Correspondence Address: Baker Botts L.L.P., 2001 Ross Avenue, Dallas, TX, 75201-2916, US

(21) Appl. No.: 09/999,051

(22) Filed: Nov. 1, 2001

Related U.S. Application Data
(60) Provisional application No. 60/245,722, filed on Nov. 2, 2000.

(51) Int. Cl. A61N 1/36

(52) U.S. Cl. 607/50

(57) ABSTRACT

A system and method for increasing the efficacy of stimulation signal therapy applied to selected tissue at a treatment site is disclosed. The method includes determining a stimulation signal capable of effecting desired results on the selected tissue and selectively modulating the stimulation signal such that the desired effect is achieved at the treatment site. The system includes a signal generator capable of generating a stimulation signal configured to produce a desired biologic effect on selected tissue at the treatment site, a modulator configured to selectively modulate the stimulation signal such that the stimulation signal correlates with at least one characteristic of the selected tissue and an emitter configured to apply the stimulation signal to the selected tissue.

### RELATED APPLICATION

This application claims priority to U.S. Provisional Patent Application Serial No. 60/245,722, filed Nov. 2, 2000, and entitled “Method and System for Modulation of Oscillating Signals to Enhance Bilogic Effects”

### TECHNICAL FIELD

The present invention relates generally to medical procedures and, more particularly, to a method and system for enhancing the efficacy of an applied tissue stimulation signal.

### BACKGROUND OF THE INVENTION

As one can readily ascertain from a simple Internet inquiry, utilizing tissue stimulation to effect healing is an area of medicine which has gained a tremendous amount of acceptance. One of the more researched areas of tissue stimulation, magnetic and electromagnetic therapy, has been measured and proven to be effective in a wide variety of applications. For example, magnetic therapy has been used to promote healing in musculoskeletal conditions including spinal fusion, fracture non-union, osteonecrosis, ligament and tendon injuries, osteoprosis, and many other conditions. Magnetic therapy has also been applied to the cardiovascular system to treat blood vessels, to stimulate tissue angiogenesis, and other applications are currently being developed.

A widely employed form of tissue stimulation utilizes pulsed electromagnetic fields (PEMF). PEMF is generally a low-energy, time varying-magnetic field commonly produced by an electromagnetic transducer coil. The electromagnetic transducer coil is situated near an injured area such that pulsing the electromagnetic transducer coil produces a driving PEMF that penetrates the underlying tissue and promotes healing of the injured area.

### SUMMARY OF THE INVENTION

While it is known and has been shown that magnetic field therapy positively affects healing processes, it is thought that the current levels of magnetic field therapy efficacy could be improved. For example, research has shown, that in a population of cells treated with PEMF therapy, only a portion of the cells in the population respond to the applied PEMF. It is believed that if the PEMF affected a greater portion of the cell population, the resultant healing processes would be more effective, i.e., the healing processes would proceed at a greater pace and encompass larger regions of tissue. In addition, increased efficacy of magnetic therapy would allow magnetic therapy devices to be engineered to take advantage of this new-found effectiveness through the use of smaller units that require less power.

It has been theorized that one of the reasons for the lack of response is that receptors on the non-responding cell in a cell population are slightly out-of-tune with cells in the remaining cell population that do respond. Thus, if a precise field is being applied to a cell population, only the cells that are in tune with the precise field will receive the field and subsequently be stimulated. The out-of-tune cells will generally not be able to receive the field and, therefore, will not be stimulated.

Therefore, there is a need for a method and system for determining and selectively modulating tissue stimulation signals capable of accomplishing a desired effect on selected tissue at a treatment site which is more efficient and more effective than current tissue stimulation signal techniques and technology. This desired effect could include either stimulation of tissue growth and healing such as would enhance fracture healing or angiogenesis. In some conditions, however, the treatment frequency might be adjusted to selectively produce tissue damage in order to dissolve a blood clot or obstructing tissue mass such as a tumor.

Thus, in accordance with teachings of the present invention, a tissue stimulation signal method and system are provided to more efficiently and more effectively produce a desired effect (either tissue growth or destruction) at a treatment site than known tissue stimulation signal methods and systems.

In one aspect, the present invention increases the efficacy of magnetic field therapy by providing a method for affecting selected tissue at a treatment site. This method includes determining at least one stimulation signal capable of affecting at least one characteristic of the selected tissue and selectively modulating at least one stimulation signal such that a desired effect on the selected tissue is achieved. Modulating the stimulation signal increases the treatment frequencies that are applied to the tissue and results in an increased number of tissue cells responding to the treatment.

In another aspect, the present invention provides a method for enhancing the biologic effects of an applied signal. The method may include generating at least one stimulation signal capable of effecting a desired result on the selected tissue at a treatment site, tuning at least one stimulation signal relative to at least one characteristic of the treatment site and applying the stimulation signal to the selected tissue at the treatment site.

In yet another aspect, the present invention provides a system for enhancing the biologic effects of signals applied to selected tissue at a treatment site including at least one signal generator capable of generating at least one stimulation signal configured to produce a desired effect on selected tissue at a treatment site, at least one modulator operably coupled to the signal generator and configured to selectively modulate at least one stimulation signal such that at least one stimulation signal correlates with at least one characteristic of the treatment site and at least one emitter, preferably coupled to at least one signal generator and at least one modulator, configured to apply at least one stimulation signal to the selected tissue at the treatment site.

In another embodiment, the present invention provides a method for enhancing the biologic effects of an applied signal. The method preferably includes generating at least one stimulation signal capable of effecting a desired result on selected tissue at a treatment site. The method then preferably selectively modulates at least one stimulation signal such that a desired effect on the tissue site is achieved and subsequently applies the stimulation signal to the selected tissue.

### BRIEF DESCRIPTION OF THE DRAWINGS

For a more complete understanding of the present invention, and the advantages thereof, reference is now made to the following written description taken in conjunction with the accompanying drawings, in which:

FIG. 1 illustrates a flow chart for one method according to teachings of the present invention;

FIG. 2 illustrates a block diagram of a system according to teachings of the present invention;

FIGS. 3A and 3B illustrate a stimulation signal routine before and after frequency modulation and the Fourier transform associated with each according to one embodiment of the present invention;

FIG. 4 illustrates a stimulation device configured for use on a shoulder according to teachings of the present invention;

FIG. 5 illustrates a stimulation device configured for use on an arm according to teachings of the present invention;

FIG. 6 illustrates a stimulation device configured for use around a leg or torso according to teachings of the present invention; and

FIG. 7 illustrates a read-out unit that may be used for displaying and recording a user's operation of a system according to teachings of the present invention.

### DETAILED DESCRIPTION OF THE INVENTION

Preferred embodiments of the present invention and its advantages are best understood by referring to the FIGS. 1-7 of the drawings, like numerals being used for like and corresponding parts of the various drawings.

FIG. 1 illustrates a flow chart indicating a method 100 of implementing a stimulation signal treatment routine capable of producing a desired effect on selected tissue at a treatment site. Method 100 begins with step 105 in which identification of the tissue to be treated is performed. This identification, or diagnosis, produces results such as a fractured bone, a tumor or growth, a blood clot, or another medical condition which may respond to stimulation signal therapy.

One of the goals of step 105 is to determine and select which type of tissue should be stimulated in order to most effectively treat the condition. For example, in the case of a fractured bone, it may be determined that it would be most effective to stimulate red blood cells and blood flow in the area of the fracture in order to enhance the calcification of the extracellular matrix, which would increase fracture healing. In the case of an organ with marginal blood supply, tissue stimulation therapy may be applied to stimulate angiogenesis (the formation of new blood vessels) in a previously ischemic area.

In the case of a blood clot, it may be determined that the most effective treatment is to use a stimulation treatment frequency that would stimulate the clot itself such that it is broken up and the blood vessel is cleared of any obstruction to blood flow. Alternatively, it might be determined that stimulating red blood cells to increase the blood flow at the site of the clot will clear the blood vessel. Therefore, a stimulation signal capable of stimulating red blood cells to increase blood flow in the clotted area may be the selected treatment.

In some cases it may be determined that stimulating certain cell or tissue types at a treatment site will be harmful to the desired outcome. In this situation, the use of certain stimulation signals should be avoided and those used should be ones which do not stimulate those particular cell and tissue types.

Once the site to be treated has been identified, including the underlying components for which the treatment will be focused, e.g., red blood cells, bone tissue, or soft tissue, method 100 proceeds to step 110 where a determination of the type of stimulation signal which will most effectively produce the desired result is made. The present invention accommodates a myriad of possible factors which can influence the type of stimulation signal used to stimulate tissue at a treatment site. For example, it may be determined in accordance with teachings of the present invention that there is a relationship between the dimensions of a particular type of cell and the wavelength or frequency of an applied PEMF to which the cell will respond. In this scenario, the cell dimensions of the tissue to be treated would be determined and an appropriate PEMF would be generated and applied to the tissue. The stimulation signal is, in effect, tuned to the frequency which will be best received by the tissue under treatment.

In addition to cell dimensions, other cell characteristics can be important in determining the most effective stimulation signal to be applied to a tissue type. For example, chemical compounds which make up or reside near the tissue can influence the type of stimulation signal which should be applied. Cell density and molecular dynamics, as well as other characteristics of a treatment site may have some bearing on what type of stimulation signal would be most effective for the treatment of the selected tissue at the treatment site.

Once the appropriate characteristics of the tissue to be treated have been determined, it can then be determined whether application of an electrical stimulation signal or a mechanical stimulation signal would be more effective in the treatment of the selected tissue. For example, consider a situation where an obstructing tissue mass needs to be broken down. If the density or other characteristics of the tissue mass indicate that an electrical stimulation signal would not effectively break the tissue down it might be desirable to use a mechanical stimulation signal, such as a sound wave, to treat the tissue mass. The mechanical stimulation signal could then be adjusted so that the resonant frequency of the tissue mass and the frequency of the mechanical stimulation signal were additive. This additive effect could then result in the over-stimulation and eventual breakdown of the tissue mass. For some applications, a combination of electrical stimulation signals and mechanical stimulation signals may be used in accordance with teachings of the present invention.

In determining the most effective stimulation signal, it may be desirable to treat more than one tissue type at a time. This will require determining a preferred stimulation signal for each tissue. For example, if it would be more effective to dissolve a blood clot by increasing blood flow and breaking up the clotting tissue, it may be determined that a sound wave stimulation signal tuned to break up the clotting tissue and a PEMF stimulation signal tuned to increase blood flow need to be simultaneously applied.

Once the most effective stimulation signal or series of signals have been determined, method 100 proceeds to step 115 at which a stimulation signal treatment plan is devised. At step 115, the most effective means of achieving the desired results on the selected tissue is further analyzed.

The most effective stimulation signal therapy routine can involve many different factors. For example, if a portion of a cell population is showing no response to an applied PEMF, it may be determined that part of the population is out of tune with the PEMF and that is why it is not responding. One method of improving the response at this point would be to tune the applied PEMF such that a larger portion of the population exhibits a response to a given frequency of stimulation.

An alternate method involves modulating the PEMF, such as by Frequency Modulation (FM) or Amplitude Modulation (AM), to effectively spread out the PEMF stimulation signal and increase the range of frequencies simultaneously applied to the tissue. This technique subsequently enables the PEMF stimulation signal to reach, be received by, and, therefore, stimulate a greater portion of the cell population.

In the situation where multiple stimulation signals are needed to treat a selected tissue site, it is in step 115 where the output routine of the stimulation signals is determined. For example, it may be decided for reasons of device or treatment efficiency that the best way for the multiple stimulation signals to be applied to the tissue site is to overlap the signals such that a first stimulation signal is applied, and at the same time a second signal is applied, and so on, up to as many signals as are necessary for an effective treatment. An alternative to this overlapping, or parallel application, of multiple stimulation signals is to transmit each of the signals serially. For example, a first signal may be transmitted for a time period, turned off and then a second stimulation signal is transmitted and then turned off. This procedure can be repeated with as many stimulation signals as are necessary for an effective treatment. The sequence can then be begun all over again starting with the first signal. Other methods of serially applying stimulation signals are considered within the scope of the present invention.

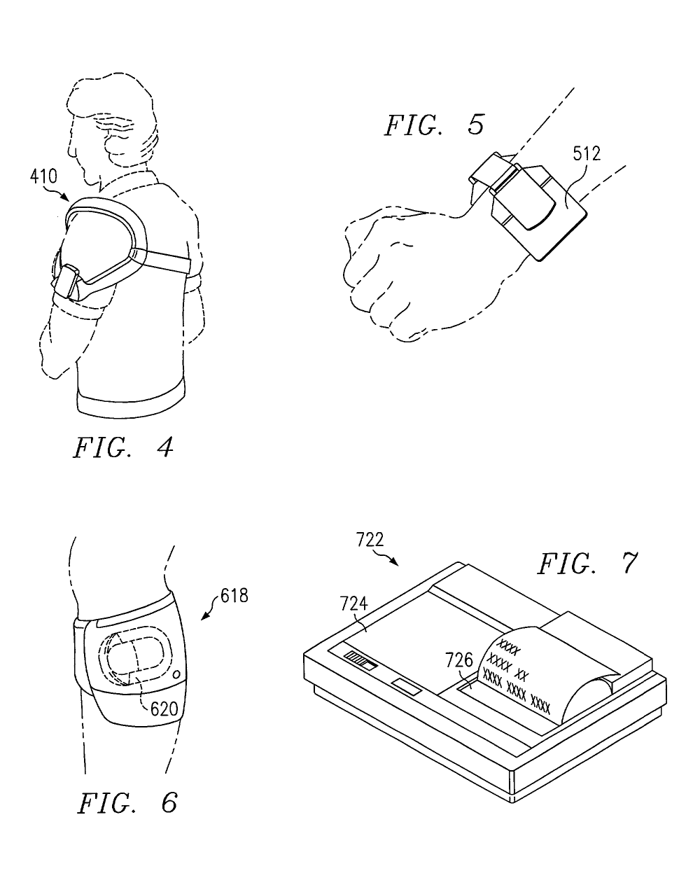

Upon determination of an appropriate stimulation signal routine, method 100 proceeds to step 120. In step 120, the stimulation signal routine is applied to the selected tissue at the treatment site. This application can take place using non-invasive devices such as those illustrated in FIGS. 4-7, discussed below, or by using devices configured to be implanted internally at or near the treatment site of the subject being treated.

To ensure the stimulation signal routine is effective, one embodiment of method 100 includes step 125 which involves monitoring the stimulation signal routine. Monitoring of the stimulation signal routine includes, but is not limited to, monitoring the effects on the selected tissue under treatment, monitoring the amount of time the stimulation signal therapy is applied, monitoring the consistency of the applied stimulation signal routine, as well as other characteristics.

One possible goal of the monitoring performed in step 125 is to provide a reference for the evaluation of the stimulation signal and the stimulation signal routine to ensure that the stimulation signal and the stimulation signal routine are producing the desired effects on the selected tissue at the treatment site. For example, if the monitoring results indicate that the stimulation signal routine has been applied as planned but laboratory and radiologic tests indicate that the selected tissue is not responding, the frequency of stimulation signal being employed would be reevaluated at step 110. If the stimulation signal in use is reaffirmed as the most effective, method 100 proceeds to step 115 for a stimulation signal routine reevaluation. Alternatively, if the monitoring results of step 125 determine that the stimulation signal routine is beginning to produce the desired results at the tissue site under treatment, method 100 returns to step 120 to continue application of the stimulation signal routine until the monitoring results are checked again. When it is determined that the results of step 125 indicate that the treatment of the selected tissue has been successful, method 100 ends stimulation signal therapy at 130.

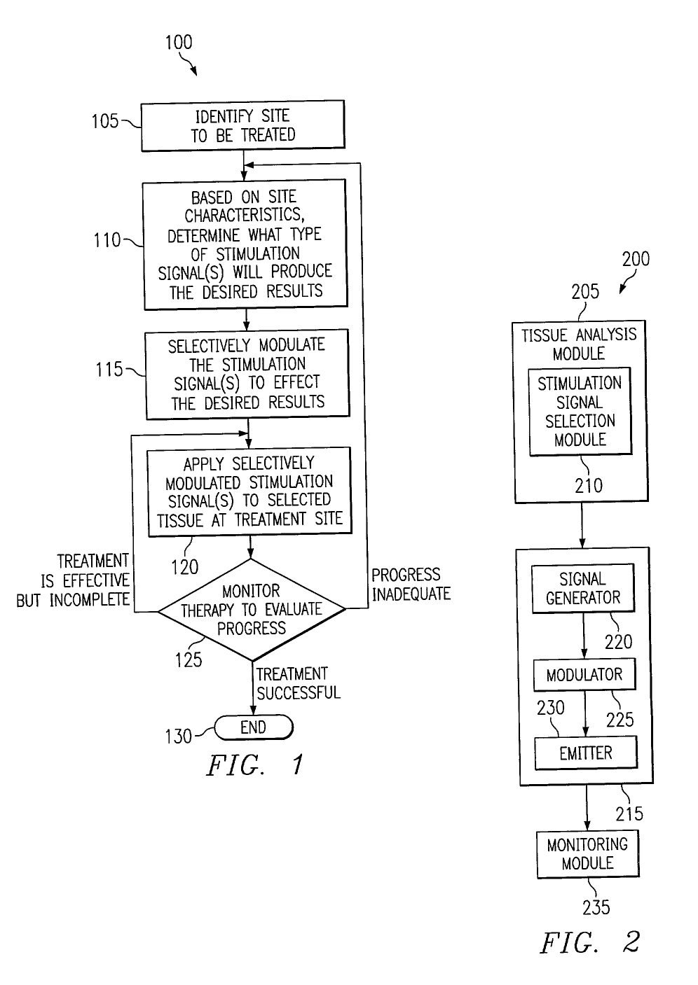

FIG. 2 illustrates a block diagram of one embodiment of a system capable of performing method 100 of FIG. 1. System 200 includes tissue analysis module 205 to enable the identification of the tissue site to be treated. Components that might be included in tissue analysis module 205 include X-ray machines, blood analyzers, chemical detection means, as well as other components for evaluating biologic effects at a treatment site. Stimulation signal selection module 210 is included in tissue analysis module 205 to allow an appropriate stimulation signal to be quickly determined. Stimulation signal module 210 might include a database consisting of scientific data supporting which type of stimulation signal is most effective on certain types of tissues, chemical compounds, cell sizes, etc. Stimulation signal module 210 might also contain simulations of the effects of various stimulation signal types on various tissue types to enable the selection of the appropriate stimulation signal for producing a desired result.

Operably coupled to tissue analysis module 205 is stimulation signal module 215. Stimulation signal module 215 includes at least one signal generator 220 capable of generating the stimulation signal determined to be appropriate by tissue analysis module 205. Signal generator 220, in a preferred embodiment, is capable of producing various waveforms with various duty cycles, amplitudes, frequencies, as well as other signal characteristics. In addition, signal generator 220 is configured with a tuning capability. Stimulation signal module 215 also includes at least one modulator 225 capable of selectively modulating the signals generated by signal generator 220. Various forms of modulation are anticipated, including, but not limited to, Frequency Modulation (FM), Amplitude Modulation (AM), duty cycle modulation, as well as variants thereof. Emitter 230 is included to enable the stimulation signal generated to be applied to the selected tissue at the treatment site.

Stimulation signal module 215 is preferably coupled to monitoring module 235. Monitoring module 235 might include memory, such as random access memory, magnetic media, as well as others, to record the stimulation signal routine being emitted by stimulation signal module 215.

Exemplary embodiments of the tissue site therapy system of the present invention are configured to provide stimulation signals, in the form of PEMFs, sound waves, or other forms of electromagnetic energy or heat energy. The treatment sites may include the shoulder, the hands, the hip, blood vessels, the heart, tumors or essentially any other anatomic region to assist in the healing of injuries or the treatment of ailments.

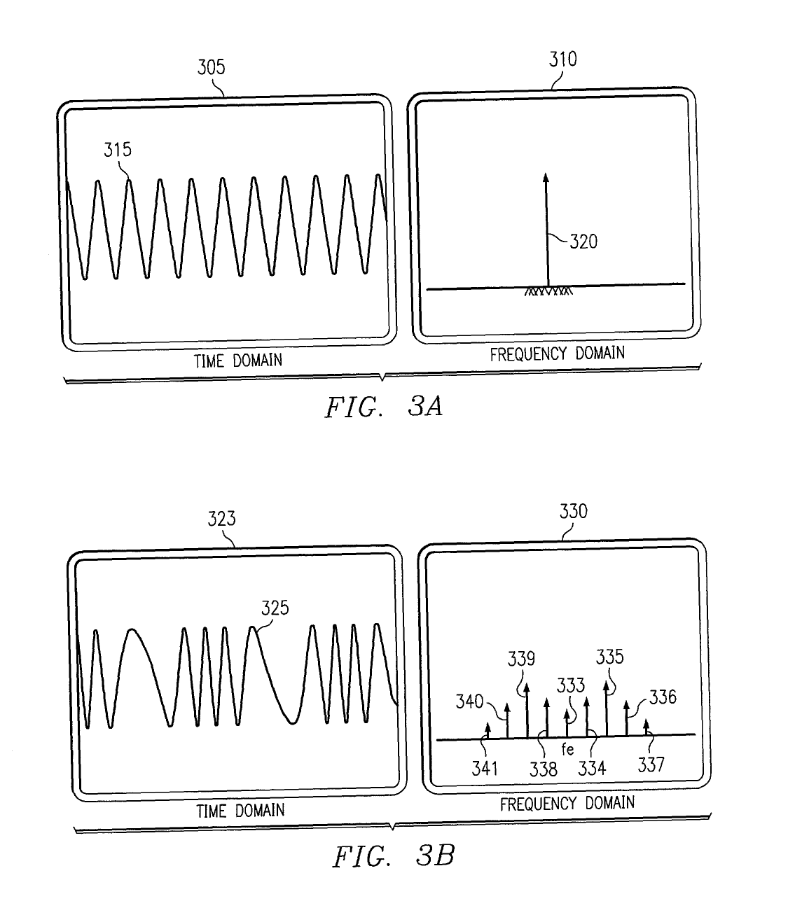

FIGS. 3A and 3B illustrate a stimulation signal routine before and after frequency modulation and the Fourier transform associated with each according to one embodiment of the present invention. Specifically, in FIG. 3A, a sinusoidal stimulation signal routine 315 is illustrated in the time domain at 305 and in the frequency domain 310. Sinusoidal stimulation signal routine 315 is oscillating at center frequency fc. As illustrated at 320, the power carried by sinusoidal stimulation signal routine 315 is centralized primarily at center frequency fc. Thus, unmodulated stimulation signal routines typically provide the majority of their power primarily at their center frequency or oscillating frequency, as illustrated at 310.

According to teachings of the present invention, a lack of response in a portion of the cells at the treatment site is likely to be displayed because the receptors of the non-responsive cells are out of tune with center frequency fc of the applied stimulation signal. Thus, the precise field of an unmodulated stimulation signal routine will be received by that portion of the cells at the treatment site which are in tune with the precise field. Accordingly, only that portion receiving the precise field will be affected by the unmodulated stimulation signal routine.

Illustrated at 323 in FIG. 3B, is the result of passing sinusoidal stimulation signal routine 315 of FIG. 3A through a frequency modulator. The resultant Fourier transform of frequency modulated stimulation signal routine 325 is illustrated at 330. One significant result of frequency modulating sinusoidal stimulation signal routine 315 is that instead of being limited to the primary power frequency fc as indicated at 320, the power in frequency modulated sinusoidal stimulation signal routine 325 is spread out over a broad range of frequencies. The result of this spreading of power is that a greater portion of the cells at the tissue site under treatment, will be effected.

As mentioned above, since the cells at the tissue site under treatment are likely to be out of tune with a primary frequency, the spreading out of the power contained in a stimulation signal routine enables a greater portions of the cells to be effected. Subsequently, as illustrated at 330, significant power can be observed not only at center frequency 333, but also at harmonics 334-337 and sub-harmonics 338-341 of frequency modulated stimulation signal routine 325. Additionally, although FIGS. 3A and 3B include a sinusoidal stimulation signal routine, stimulation signal routines of other forms, such as square, triangular, diamond and other, are also considered within the scope of the present invention.

FIGS. 4-7 illustrate different examples of non-invasive stimulation therapy systems formed according to teachings of the present invention. The stimulation signal generators employed to effect the present invention may be formed and anatomically contoured for the shoulder, the wrist, the hip or other areas of the anatomy. FIG. 4, in particular, shows a contoured triangular stimulation signal transducer 410 that is anatomically contoured for providing stimulation therapy to the shoulder area. That is, one side is curved to fit over the top of the shoulder so that corresponding angular areas are positioned in front and in back of the shoulder, with the other sides being curved down along the upper arm. The shoulder transducer is an integral unit including drive electronics and control electronics that may be held in place by a body strap.

FIG. 5 shows a placement of a stimulation therapy device that includes a stimulation transducer 512 according to the teachings of the present invention, but of a size and shape that best suits the patient's wrist or other limb portion. Stimulation transducer drive circuitry and control electronics are preferably included as an integral part of stimulation transducer 512.

FIG. 6 shows yet another embodiment of the present invention as a hip belt stimulation therapy device 618 that a patient may wear around the waist, the stimulation transducer 620 arranged over the hip area. The drive electronics and control circuitry, again, are an integral part of stimulation therapy device 618.

FIG. 7 shows a read-out unit 722 that may be used for displaying and recording a patient's operation of the present invention. The present invention may include, therefore, an extended memory and built-in printer interface 724 for providing the ability to correlate patient usage with desired healing progress and provide results on a paper print-out device 726. The system of the present embodiment, for example, may store months of compliance data for developing important correlation data and print out such data using a paper print-out device 726.

Although the present invention and its advantages have been described in detail it should be understood that various changes, substitutions, and alterations can be made hereto without departing from the spirit and scope of the invention as defined by the following claims.

What is claimed is:

1. A method for stimulating tissue growth comprising:
determining at least one stimulation signal capable of affecting at least one characteristic of selected tissue at the treatment site; and
selectively modulating the at least one stimulation signal such that the desired effect on the selected tissue is enhanced.

2. The method of claim 1, further comprising correlating at least one characteristic of the stimulation signal with at least one characteristic of the selected tissue.

3. The method of claim 1, wherein the at least one characteristic of the stimulation signal is frequency.

4. The method of claim 1, wherein the range of frequencies is 60 Hz to 30 kHz.

5. The method of claim 1, wherein the optimal frequency for stimulating spinal tissue is 3400 Hz.

6. The method of claim 1, wherein selectively modulating comprises frequency modulating or amplitude modulating the at least one stimulation signal.

7. The method of claim 1, wherein the at least one characteristic of the selected tissue is based on cell structure.

8. The method of claim 1, wherein the desired effect comprises structural deformation of the treatment site such that the selected tissue is selectively damaged.

9. The method of claim 1, wherein selectively modulating comprises overlapping a plurality of stimulation signals capable of affecting at least one characteristic of the selected tissue.

10. The method of claim 1, wherein at least one characteristic of the selected tissue is chemical composition of a selected cell type.

11. The method of claim 1, wherein selectively modulating comprises sequential application of stimulation signals capable of affecting at least one characteristic of the selected tissue.

12. A method for using PEMF to dissolve tissue masses comprising:
determining at least one stimulation signal capable of affecting at least one characteristic of selected tissue at the treatment site;
selectively modulating the at least one stimulation signal such that the selected tissue is maximally damaged; and
adjusting the selected stimulation signal and modulation based on results of monitoring the treatment site for response to the at least one stimulation signal with laboratory and radiologic studies.

13. The method of claim 12, further comprising correlating at least one characteristic of the stimulation signal with at least one characteristic of the selected tissue.

14. The method of claim 12, wherein multiple cell characteristics are correlated with the frequency and pulsed interval of the stimulation signal.

15. The method of claim 12, wherein selectively modulating comprises frequency modulating or amplitude modulating the at least one stimulation signal to maximize tissue destruction.

16. The method of claim 12, wherein the at least one characteristic of the selected tissue is based on the specific cell type architecture.

17. The method of claim 12, wherein the desired effect comprises structural deformation of specific cell types at the treatment site such that the selected tissue is selectively damaged and dissolved.

18. The method of claim 12, wherein selectively modulating further comprises adjusting a frequency of the at least one stimulation signal to dissolve a tissue mass associated with a tumor.

19. The method of claim 12, wherein at least one characteristic of the selected tissue is chemical composition of the specific cell type.

20. The method of claim 12, wherein selectively modulating comprises sequential application of stimulation signals capable of affecting the at least one characteristic of the selected tissue.

21. A system for enhancing biologic effects of signals applied at a treatment site comprising:
at least one signal generator capable of generating at least one stimulation signal configured to produce a desired biologic effect on selected tissue at the treatment site;
at least one modulator operably coupled to the signal generator and configured to selectively modulate the at least one stimulation signal such that the at least one stimulation signal correlates with at least one characteristic of the selected tissue; and
at least one emitter, operably coupled to at least one signal generator and the at least one modulator, to apply at least one stimulation signal to the selected tissue.

22. The system of claim 21, further comprising a monitoring device operably coupled to the emitter and capable of monitoring the stimulation signal applied by the emitter.

23. The system of claim 21, further comprising a tissue analysis module operably coupled to the signal generator and configured to determine at least one characteristic of the selected tissue at the treatment site.

24. The system of claim 21, wherein the signal generator comprises a pulsed electromagnetic field stimulation signal generator.

25. The system of claim 21, wherein the signal generator comprises a sound wave stimulation signal generator.

26. The system of claim 21, further comprising the emitter operable to be implanted into the treatment site.

27. The system of claim 21, further comprising an electromagnetic signal generator and a sound wave signal generator.

28. A method for enhancing the biologic effects of an applied stimulation signal comprising:
determining at least one stimulation signal capable of effecting a desired result on at least one characteristic of a selected tissue at a treatment site;
generating the at least one stimulation signal capable of effecting the desired result at a treatment site;
tuning the at least one stimulation signal relative to at least one characteristic of the selected tissue; and
applying the stimulation signal to the selected tissue.

29. The method of claim 28 further comprising modulating the at least one stimulation signal.

30. The method of claim 28 further comprising selectively modulating the at least one stimulation signal such that application of the selectively modulated stimulation signal produces the desired result on the selected tissue at the treatment site.

31. The method of claim 28 further comprising modulating the frequency and the amplitude of the stimulation signal.

32. A method for enhancing the biologic effects of an applied signal comprising:
generating at least one stimulation signal capable of effecting a desired result on selected tissue at a treatment site;
selectively modulating at least one stimulation signal such that a desired effect on the selected tissue is achieved; and
applying the stimulation signal to the selected tissue.

33. The method of claim 32 further comprising modulating the frequency of the at least one stimulation Signal.

34. The method of claim 32 further comprising overlapping a plurality of stimulation signals such that application of the stimulation signals produce the desired result on the selected tissue at the treatment site.

35. The method of claim 32 further comprising determining at least one stimulation signal capable of affecting at least one characteristic of the selected tissue at the treatment site.

36. A method of dissolving clots in blood vessels comprising:
determining at least one stimulation signal capable of affecting at least one characteristic of selected tissue at the treatment site;
selectively modulating the at least one stimulation signal such that the clots are maximally damaged; and
monitoring the treatment site for response to the at least one stimulation signal with laboratory and radiologic studies.

37. A system for dissolving a tumor comprising:
at least one signal generator capable of generating at least one stimulation signal configured to produce a desired biologic effect on selected tissue at the treatment site;
at least one modulator operably coupled to the signal generator and configured to selectively modulate the at least one stimulation signal such that the at least one stimulation signal correlates with at least one characteristic of the selected tissue; and
at least one emitter, operably coupled to at least one signal generator and the at least one modulator, to apply at least one stimulation signal to the selected tissue.
